(19) United States
(12) Patent Application Publication
(10) Pub. No.: US 2025/0329456 A1
(43) Pub. Date: Oct. 23, 2025

(54) SYSTEM AND METHOD FOR GENERATIVE ARTIFICIAL INTELLIGENCE (AI) MODEL FINETUNING FOR CLINICAL WORKFLOWS

(71) Applicant: Microsoft Technology Licensing, LLC, Redmond, WA (US)

(72) Inventors: Noel Christopher Codella, Redmond, WA (US); Matthew Preston Lungren, Palo Alto, CA (US); Neil Tenenholtz, Cambridge, MA (US)

(21) Appl. No.: 18/639,571

(22) Filed: Apr. 18, 2024

(51) Int. Cl. G16H 50/20; G16H 15/00; G16H 30/40

(52) U.S. Cl. CPC G16H 50/20; G16H 15/00; G16H 30/40

(57) ABSTRACT

A method, computer program product, and computing system for generating an internal state prompt with medical content and a multi-action task to perform on a healthcare system. A first output healthcare system command is generated by processing the internal state prompt using a trained multimodal generative artificial intelligence (AI) model. The first output healthcare system command is converted into a first healthcare system-executable command associated with the multi-action task for a first target healthcare subsystem. Modified medical content is generated by executing the first healthcare system-executable command on the medical content using the first target healthcare subsystem. The internal state prompt is updated with the modified medical content generated by executing the first healthcare system-executable command and the first output healthcare system command listed as a past action performed during execution of the multi-action task.

### BACKGROUND

Generative artificial intelligence models have demonstrated the ability to leverage a text-based web-browser to explore web data to retrieve relevant information to better answer posed questions. For example, an interface exposes a certain set of commands to generative AI models and human users alike (i.e., “search <query>”, “click on <link>”, “scroll down/up”, “quote <text>”, and “end: answer”) that performs some action within the web browser which would then return response data in the form of a current state record (otherwise known as a “prompt”). After collecting example questions, actions, and answers from human users using the web browser, the generative AI model is finetuned to mimic those humans.

Other works have demonstrated generative AI model's ability to leverage application programming interfaces (APIs), and coding libraries to solve open domain tasks. However, even as multiple systems are connected, these generative AI models are unable to continually interact with other multimodal systems to explore, obtain, or modify multimodal data (e.g., image, text, structured, or chart-based medical content across multiple healthcare subsystems) as part of a larger task involving multiple steps or actions across numerous modalities.

### BRIEF DESCRIPTION OF THE DRAWINGS

FIG. 1 is a flow chart of one implementation of a generative AI model clinical process;

FIG. 2 is a diagrammatic view of the generative AI model clinical process of FIG. 1 during the processing of a first action of a multi-action task using a trained multimodal generative AI model;

FIG. 3 is a diagrammatic view of the generative AI model clinical process of FIG. 1 during the processing of a second action of a multi-action task using the trained multimodal generative AI model;

FIG. 4 is a flow chart of one implementation of a generative AI model clinical process during training of a multimodal generative machine learning model; and

FIG. 5 is a diagrammatic view of computer system and generative AI model clinical process coupled to a distributed computing network.

Like reference symbols in the various drawings indicate like elements.

### DETAILED DESCRIPTION OF THE PREFERRED EMBODIMENTS

Implementations of the present disclosure enable a multimodal generative AI model to interact with healthcare subsystems (e.g., radiology imaging systems; picture archiving and communication system (PACS); electronic health record (EHR) databases; vendor neutral archive databases; and/or other machine learning models) to iteratively process medical content, perform actions within a healthcare environment, and to accomplish multi-action tasks. For example, in the context of automated medical image report generation and other healthcare services, implementations of the present disclosure define a set of commands that carry out actions on healthcare subsystems (e.g., multimodal medical database systems) that a multimodal generative AI model can leverage to explore patient data (including medical image content), carry out tasks, write reports, order or recommend new clinical tests, and/or make diagnostic recommendations. Accordingly, the described generative AI model clinical process allows for multimodal generative AI models to interact with the data (i.e., medical content) and healthcare subsystems. This enables the multimodal generative AI model to access and “explore” patient data—imaging information, medical record information, sequential data, medical guidelines, and more, to facilitate analysis of patients and the automated generation of medical image reports, ordering of new clinical tests, and/or processing of billing codes.

Generally, conventional AI models for medical imaging take as input a fixed number of images and other information regarding a given clinical scenario, producing fixed outputs or dialogue engines over the mentioned fixed inputs. However, these models are unable to iteratively request specific additional information necessary to achieve a given task, or to explore a more open medical data space defined by healthcare subsystems. For example, these conventional AI models are unable to communicate with various healthcare subsystems to obtain or modify medical information. In contrast, the generative AI model clinical process described in this disclosure enables a multimodal generative AI model to perform a series of actions to accomplish a multi-action task concerning the entire patient record and to make automated decisions as to what data is necessary to process to accomplish the task. The model is also enabled to fully explore not only a single three-dimensional dataset, but multiple three-dimensional datasets over time, or multimodal datasets within the same session in coordination with various healthcare subsystems.

For example, the generative AI model clinical process generates an internal state prompt with medical content and a multi-action task to perform on a healthcare system. The internal state prompt is a structured prompt that includes medical content to process, a task to perform, and any past actions performed. The internal state prompt is provided to a trained multimodal generative AI model to generate a first output healthcare system command, which is generated by processing the internal state prompt. The first output healthcare system command describes an interaction with a particular healthcare subsystem to modify the medical content, obtain additional medical content, alert a healthcare provider, generate a medical treatment plan, generate a prescription, etc. The trained multimodal generative AI model generates a next action to perform in the multi-action task.

The first output healthcare system command is converted into a first healthcare system-executable command associated with the first action for a first target healthcare subsystem. For example, a predefined healthcare system-executable command is identified for the first output healthcare system command from a plurality of predefined healthcare system-executable commands. The predefined healthcare system-executable commands are mapped to output healthcare system commands from the multimodal generative AI model to describe how a respective healthcare subsystem performs a particular operation on the medical content using inputs provided by the multimodal generative AI model output. Modified medical content is generated for the multi-action task by executing the first healthcare system-executable command on the medical content using the first target healthcare subsystem. The internal state prompt is updated with the modified medical content and the first output healthcare system command listed as a past action performed during execution of the multi-action task. In this manner, generative AI model clinical process 10 iteratively processes and modifies medical content by performing individual actions sequentially as defined by the internal state prompt, processing the internal state prompt using the multimodal generative AI model to generate an output for a healthcare subsystem and a next action, converting the output of the multimodal generative AI model into a command executable by the respective healthcare subsystem, modifying the medical content using the healthcare subsystem, and updating the internal state prompt with the modified medical content and the first output healthcare system command listed as a past action performed during execution of the multi-action task. This process is repeated until the multi-action task is marked as completed by the generative AI model.

The details of one or more implementations are set forth in the accompanying drawings and the description below. Other features and advantages will become apparent from the description, the drawings, and the claims.

### The Generative AI Model Clinical Process:

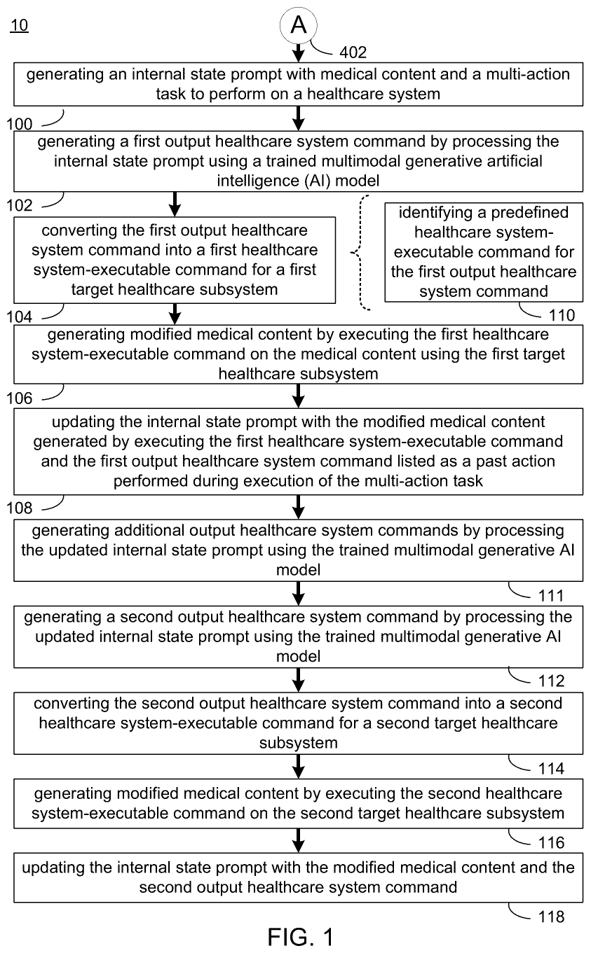

Referring to FIGS. 1-5, generative AI model clinical process 10 generates 100 an internal state prompt with medical content and a multi-action task to perform on a healthcare system. A first output healthcare system command is generated 102 by processing the internal state prompt using a trained multimodal generative artificial intelligence (AI) model. The first output healthcare system command is converted 104 into a first healthcare system-executable command associated with the multi-action task for a first target healthcare subsystem. Modified medical content is generated 106 by executing the first healthcare system-executable command on the medical content using the first target healthcare subsystem. The internal state prompt is updated 108 with the modified medical content generated by executing the first healthcare system-executable command and the first output healthcare system command listed as a past action performed during execution of the multi-action task.

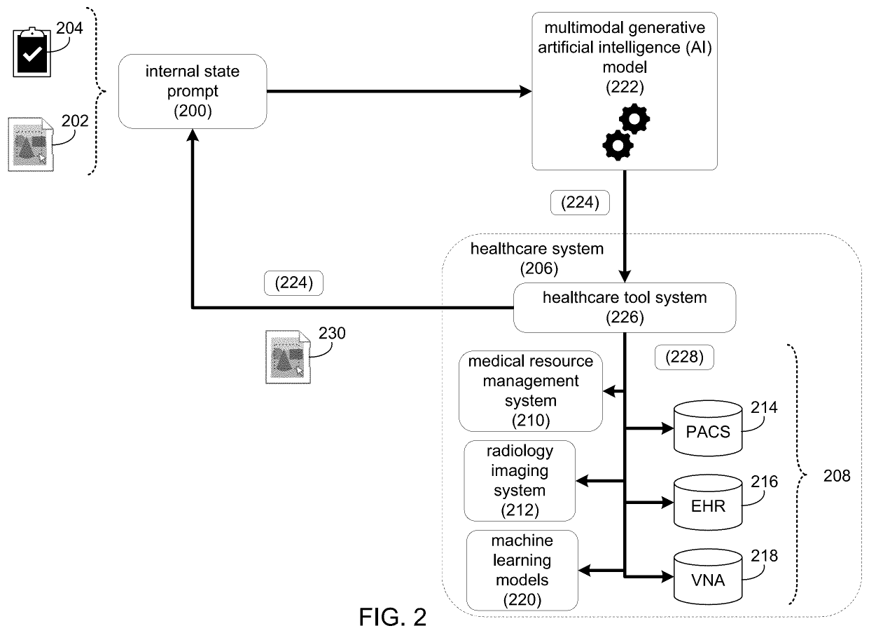

In some implementations, generative AI model clinical process 10 generates 100 an internal state prompt with medical content and a multi-action task to perform on a healthcare system. Referring also to FIG. 2 and in some implementations, an internal state prompt (e.g., internal state prompt 200) is generated 100 using medical content (e.g., medical content 202) and a multi-action task (e.g., multi-action task 206) to perform on a healthcare system (e.g., healthcare system 208). For example, a user selects (e.g., using a user interface) a particular multi-action task to perform and by selecting or uploading medical content 202. Internal state prompt 200 is generated by populating a template as shown below. As shown in FIG. 2, healthcare system 208 includes a plurality of healthcare subsystems (e.g., healthcare subsystems 210). In the example of FIG. 2, healthcare subsystems 210 include a medical resource management system that allows for the accessing and scheduling of patient visits, fulfillment of prescriptions, and/or the scheduling of patient treatment (e.g., medical resource management system 210); a radiology imaging system (e.g., radiology imaging system 212); a picture archiving and communication system (PACS) (e.g., PACS 214); an electronic health record (EHR) database (e.g., EHR database 216); a vendor neutral archive database (VNA) (e.g., VNA database 218); and/or other machine learning models (e.g., machine learning models 220). In some implementations, healthcare subsystems 210 process, generate, and modify medical content (e.g., radiology imaging system 212 generates medical image content which is stored in VNA database 218 using PACS 214). However, managing these separate healthcare subsystems is limited to distinct clinical workflows where data is processed and passed manually through separate users of each healthcare subsystem. Accordingly and as will be discussed in greater detail below, using internal state prompt 200, medical content 202 is iteratively processed for multi-action task 206 by a multimodal generative AI model (e.g., multimodal generative AI model 222) to generate an output healthcare system command that is converted into a healthcare system-executable command by one of healthcare subsystems 210 to perform a multi-action task on medical content 202.

In some implementations, multi-action task 206 is a series of steps or actions to be performed using medical content 202 to fulfill a particular purpose. For example, medical content can be processed for indication of a particular medical issue (e.g., processing a CT scan of a patient's head to identify signs of a suspected stroke). In some implementations, the multi-action task includes a series of actions to process medical content for a diagnostic-based task. In one example, multi-action task 206 is a predefined task that is selectable by a user (e.g., using a graphical user interface) and predefined for the trained multimodal generative AI model. For example and as will be discussed in greater detail below, generative AI model clinical process 10 trains multimodal generative AI model 222 to perform particular predefined sets of tasks where the tasks are performed by a user. Each action is recorded and used to generate multiple actions for the multi-action task. In this manner, when a predefined multi-action task is selected for internal state prompt 200, multimodal generative AI model 222 is trained to perform the multiple actions for the multi-action task by sequentially processing each action and performing the associated processing of the medical content required by the multi-action task.

In some implementations, medical content 202 includes medical information concerning an individual's health and/or treatment; health records; radiological images, computed tomography (CT) scans; X-rays, and/or treatment plans. In one example, medical content 202 includes medical image content. For example, medical image content includes radiological images, X-ray images, CT scans, MRI images, ultrasound images, positron emission tomography (PET) scans, fluoroscopy images, endoscopy images, and other types of images associated with a patient's health. In some implementations, many clinical workflows include the analysis of medical image content. However, conventional approaches that attempt to use artificial intelligence are limited to performing individual medical image processing with transitions between medical images for different medical features (i.e., anatomical features including organs, tissue, bones, etc.) performed by human users. As such, conventional approaches are unable to process multi-action tasks that modify the medical image content and are unable to access resources or functionality of healthcare subsystems when processing multi-action tasks.

As shown in FIG. 2, generative AI model clinical process 10 generates 100 internal state prompt 200 with medical content 202 and multi-action task 206 to perform on healthcare system 208 by processing a selection of multi-action task 206 and populating internal state prompt 200 with a description of multi-action task 206 and medical content 202 (or a reference to medical content 202). For example, suppose a user desires to process a head CT scan for indication of a suspected stroke. In this example, generative AI model clinical process 10 receives a selection of a multi-action task (e.g., multi-action task 206) for identifying an indication of a suspected stroke. An example of the template of internal state prompt 200 for multi-action task 206 is shown below:
Task: Indication suspected stroke.
Instruction: Generate findings, coordinate care management.
Quotes: [None]
Past Actions: [None]
List of input data:
   Current Image: <Slice 1> <image tokens>
   Organ regions: Cerebellum: <bounding box coordinates>, Cerebrum: <bounding box coordinates>, Brainstem: <bounding box coordinates>, . . .
   EHR: <Patient labs and clinical notes>
Actions Left: 100
Next Action:

As shown in the above example of internal state prompt 200 and in some implementations, generative AI model clinical process 10 generates 100 internal state prompt 200 with a list of input data including the medical content (e.g., medical image content from the CT scan, a description of bounding boxes for multiple medical features (e.g., organs) generated by a separate AI model, and a medical report from EHR database 216), past actions, a number of remaining actions, and a next action. In this example, the number of remaining actions is a predefined, default value and/or a value defined specifically for multi-action task 206. As this is the first action, no “past action” is included.

In some implementations, generative AI model clinical process 10 generates 102 a first output healthcare system command by processing the internal state prompt using a trained multimodal generative artificial intelligence (AI) model. A multimodal generative AI model (e.g., multimodal generative AI model 222) is configured to receive input prompts including text and/or images, example entries, and/or contextual information concerning a request to generate an audio response. Multimodal refers to the ability of the generative AI model to understand and generate content in different forms (e.g., text and images). Multimodal generative AI model 222 includes a neural network with many parameters (typically millions or billions of weights or more), trained on large quantities of unlabeled and/or labeled data using self-supervised learning, semi-supervised learning, and/or fine-tuning of the weights to cater the neural network for particular tasks or workloads. In some implementations, multimodal generative AI model 222 is one of a multimodal large language model (LLM) and a large multimodal model (LMM). In one example, multimodal generative AI model 222 is the GPT-4V LLM from OpenAIR (i.e., GPT-4 with vision (GPT-4V) that enables users to instruct the multimodal generative AI model to analyze image inputs provided by the user). In some implementations, first output healthcare system commands may involve physical interaction with the patient or impact treatment plans. In such cases, approval and/or feedback from healthcare providers may be requested through a separate healthcare system command.

In the example of FIG. 2, with internal state prompt 200 as an input, multimodal generative AI model 222 processes internal state prompt 200 and outputs a first output healthcare system command (e.g., first output healthcare system command 224). Output healthcare system command 224 is a command generated by multimodal generative AI model 222 that indicates a command to be performed by a healthcare subsystem (e.g., healthcare subsystems 210) of healthcare system 208. For example, suppose multimodal generative AI model 222 generates 102 an output of “Change Slice Position to Slice 12”. In this example, generative AI model clinical process 10 provides first output healthcare system command 224 associated with changing the slice position to “slice 12” to healthcare system 208 for processing.

In some implementations, multimodal generative AI model 222 may not always generate a first output healthcare system command but may generate modified medical content directly. For example, suppose multimodal generative AI model 222 generates 102 an output of “Change Slice Position to Slice 12” where multimodal generative AI model 222 is able to change the slice position to slice 12. In this example, generative AI model clinical process 10 provides the modified medical content with slice 12 for processing in a next action. As will be discussed in greater detail below, the next action is determined by multimodal generative AI model 222 for further executing multi-action task 206 based on internal state prompt 200 (e.g., a first output healthcare system command performed, medical content 202, and any other information defined for internal state prompt 200).

In some implementations, generative AI model clinical process 10 converts 104 the first output healthcare system command into a first healthcare system-executable command associated with the first action for a first target healthcare subsystem. For example, first output healthcare system command 224 is an output generated by multimodal generative AI model 222 that describes a command for a target healthcare subsystem to perform. In this example, generative AI model clinical process 10 provides first output healthcare system command 224 to a healthcare tool system (e.g., healthcare tool system 226) that interfaces with healthcare subsystems 210 to effectuate first output healthcare system command 224. Accordingly, generative AI model clinical process 10 uses healthcare tool system 226 to convert or map first output healthcare system command 224 (that is not executable by a target healthcare subsystem) to a first healthcare system-executable command (e.g., first healthcare system-executable command 228). First healthcare system-executable command 228 is a command that is executed on a respective target healthcare subsystem (by healthcare tool system 226). For example, first healthcare system-executable command 228 is configured to be processed and executed by a target healthcare subsystem (the healthcare subsystem that healthcare tool system 226 determines to be associated with first output healthcare system command 224).

In some implementations, converting 104 the first output healthcare system command into the first healthcare system-executable command includes identifying 110 a predefined healthcare system-executable command for the first output healthcare system command from a plurality of predefined healthcare system-executable commands. For example, first output healthcare system command 224 includes a description of the action to be performed by a healthcare subsystem but may not include the particular formatting to execute the action on the healthcare subsystem. Accordingly and in some implementations, generative AI model clinical process 10 converts 104 first output healthcare system command 224 into first healthcare system-executable command 228 by identifying 110 a predefined healthcare system-executable command from a plurality of predefined healthcare system-executable commands. Examples of descriptions of output healthcare system commands and corresponding descriptions of healthcare system-executable commands are shown below in Table 1.

TABLE 1

| Output Healthcare System | Healthcare System-executable |
|---|---|
| Command Description | Command Description |
| Adjust window center up/down N | Changes window leveling center |
| units | up or down |
| Adjust window width larger or | Changes window leveling width |
| smaller N units | wider or narrower |
| Adjust window center and position | Changes window levels to establish |
| to presets for modality <X> | presets for certain imaging |
| | modalities |
| Query system of record for | Retrieves text-based information |
| available clinical data containing | from patient's record |
| fields <list of fields> spanning | |
| date range <range> | |
| Change imaging plane orientation | Rotates and otherwise changes |
| to <orientation> | orientations of images |
| Get HU of target region in current | Measures Houndsfield units of a |
| image <X1, Y1> <X2, Y2> | region of the image |
| Get physical distance between two | Measures physical distances between |
| points <X1, Y1> <X2, Y2> | points |
| Instantiate secondary AI model or | Calls up a secondary model to tool to |
| measurement tool on current | generate distilled information about |
| series <AI model> <list of series> | the image |
| Query medical standards reference | Calls up information from establish |
| for guidelines and best practices | medical practice guidelines |
| for <topic summary> | |
| Activate clinical communications | Notifies physicians of high priority |
| system to alert physician of | findings |
| findings <findings> | |
| Query system of record for prior | Retrieves prior reports on this |
| reports | patient |
| Change slice position up/down N | Changes the slice position and |
| units | updates the current image |
| Get a list of series available in | Provides a list of the available |
| an imaging study | imaging |
| Change series in imaging study | Change the displayed series |
| to <series> | |
| Summarize and generate reference | Generate a text summary of the |
| based on observations | current image and add this as a |
| | reference, including slide number |
| | and any relevant groundings. |
| Generate findings | Generate the findings of the report |
| Generate impression | Generate the impression of the report |
| End: finalize report | Properly format the entire report |

As shown in the descriptions of Table 1, generative AI model clinical process 10 converts 104 first output healthcare system command 224 into first healthcare system-executable command 228 by identifying 110 a predefined healthcare system-executable command from a plurality of predefined healthcare system-executable commands. For example, the plurality of predefined healthcare system-executable commands are stored in a database with executable logic and/or reference to APIs associated with each respective healthcare subsystem. Accordingly and in response to receiving a first output healthcare system command, healthcare tool system 226 identifies a corresponding first healthcare system-executable command (e.g., as shown in Table 1). For example and as will be described in greater detail below, generative AI model clinical process 10 is trained to output specific first output healthcare system commands that healthcare tool system 226 processes to identify a corresponding first healthcare system-executable command. Accordingly, generative AI model clinical process 10 converts 104 first output healthcare system command 224 into first healthcare system-executable command 228 by performing a textual comparison between the output of multimodal generative AI model 222 (i.e., first output healthcare system command 224) and identifying 110 a corresponding predefined healthcare system-executable command (e.g., as shown in Table 1). In some implementations, the plurality of predefined healthcare system-executable commands are generated by users and/or using a generative AI model. Accordingly, it will be appreciated that predefined healthcare system-executable commands can be added or modified continually to address changes in target healthcare subsystems and/or multimodal generative AI model 222.

In some implementations, generative AI model clinical process 10 generates 106 modified medical content by executing the first healthcare system-executable command on the medical content using the first target healthcare subsystem. For example, using target healthcare subsystem 210, generative AI model clinical process 10 generates modified medical content by executing first healthcare system-executable command 228. Returning to the above example where multimodal generative AI model 222 generates 106 an output of “Change Slice Position to Slice 12”, generative AI model clinical process 10 converts this output healthcare system command into first healthcare system-executable command 228 which directs a healthcare subsystem to change the slice position to slice 12 and updates the medical image content. In this example, generative AI model clinical process 10 generates 106 modified medical content (i.e., slice position to slice 12). In this example, generative AI model clinical process 10 (using healthcare tool system 226) to provide modified medical content 230 and second action 226 to internal state prompt 200 for updating.

In some implementations, generative AI model clinical process 10 updates 108 the internal state prompt with the modified medical content generated by executing the first healthcare system-executable command and the first output healthcare system command listed as a past action performed during execution of the multi-action task. For example, updating 108 internal state prompt 200 includes revising the entries of internal state prompt 200 with the results from multimodal generative AI model 222 and healthcare subsystems 210 including modified medical content. Continuing with the above example, generative AI model clinical process 10 updates 108 internal state prompt 200 as follows:
Task: Indication suspected stroke.
Instruction: Generate findings, coordinate care management.
Quotes: [None]
Past Actions:
   Change Slice Position to Slice 12
List of input data:
   Current Image: <Slice 12> <image tokens>
   Organ regions: Cerebellum: <bounding box coordinates>, Cerebrum: <bounding box coordinates>, Brainstem: <bounding box coordinates>, . . .
   EHR: <Patient labs and clinical notes>
Actions Left: 100
Next Action:

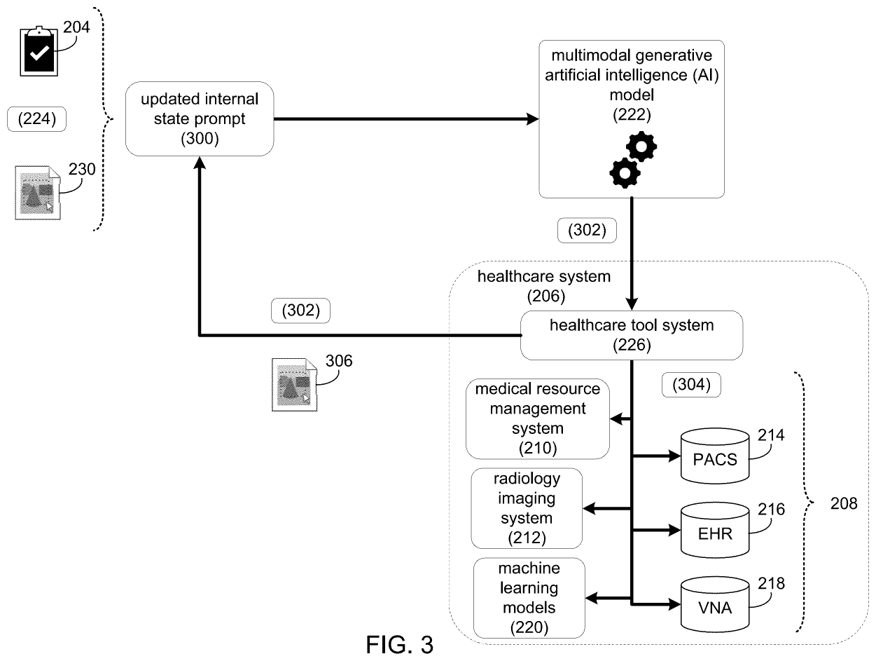

As shown above, the “past actions” include first output healthcare system command 224 and the “current image” include modified medical content 230 concerning slice 12. In some implementations and referring also to FIG. 3, updated internal state prompt 300 includes the information needed for processing a next action for processing slice 12.

In some implementations, generative AI model clinical process 10 generates 111 additional output healthcare system commands by processing the updated internal state prompt using the trained multimodal generative AI model with the first output health as context for generating the additional output healthcare system commands to perform the multi-action task. For example, internal state prompt 200 provides a history of past actions performed by multimodal generative AI model 222 to establish context and ensure that future requests made of multimodal generative AI model 222 continue to process subsequent actions of multi-action task 206 by generating additional output healthcare system commands that perform individual actions of multi-action task 206.

In some implementations, generating 111 additional output healthcare system commands includes generating 112 a second output healthcare system command by processing the updated internal state prompt using the trained multimodal generative AI model. For example, generative AI model clinical process 10 processes updated internal state prompt 300 using trained multimodal generative AI model 222 to generate 112 a second output healthcare system command (e.g., second output healthcare system command 302). Returning to the above example, multimodal generative AI model 222 generates 112 second output healthcare system command 302 for summarizing and referencing a subdural hemorrhage (i.e., “Summarize and reference: ‘subdural hemorrhage’ [slice 12]”).

In some implementations, generative AI model clinical process 10 converts 114 the second output healthcare system command into a second healthcare system-executable command associated with the multi-action task for a second target healthcare subsystem. For example, generative AI model clinical process 10 converts 114 second output healthcare system command 302 into a second healthcare system-executable command (e.g., second healthcare system-executable command 304) using healthcare tool system 226 by identifying 110 a predefined healthcare system-executable command from the plurality of predefined healthcare system-executable commands. In this example, generative AI model clinical process 10 converts 114 second output healthcare system command 302 into second healthcare system-executable command 304 for a target healthcare subsystem that provides information to summarize and reference the “subdural hemorrhage” identified by multimodal generative AI model 222.

In some implementations, generative AI model clinical process 10 generates 116 modified medical content by executing the second healthcare system-executable command on the second target healthcare subsystem. For example, using target healthcare subsystem 210, generative AI model clinical process 10 generates modified medical content by executing second healthcare system-executable command 304. Returning to the above example where multimodal generative AI model 222 generates 112 an output of “Summarize and reference: ‘subdural hemorrhage’ [slice 12]”, generative AI model clinical process 10 converts 114 this output healthcare system command into second healthcare system-executable command 304 which directs a healthcare subsystem to summarize and reference a subdural hemorrhage in slice 12. In this example, generative AI model clinical process 10 generates 116 modified medical content (i.e., summary and description of the identified subdural hemorrhage). Generative AI model clinical process 10 (using healthcare tool system 226) provides modified medical content 306 and second output healthcare system command 302 to updated internal state prompt 300 for updating.

In some implementations, generative AI model clinical process 10 updates 118 the internal state prompt with the modified medical content generated by executing the second healthcare system-executable command associated with the second action. As discussed above, generative AI model clinical process 10 continually updates internal state prompt 300 for each subsequent action. For example, generative AI model clinical process 10 updates 118 internal state prompt with records of analysis that are generated using the healthcare system-executable command “Summarize and generate reference based on observations”. Continuing with the above example, generative AI model clinical process 10 updates 118 internal state prompt 300 as follows:
Task: Indication suspected stroke.
Instruction: Generate findings, coordinate care management.
Quotes:
   Slice 12: “subdural hemorrhage”
Past Actions:
   Change Slice Position to Slice 12
   2. Summarize and reference: “subdural hemorrhage” [slice 12]
List of input data:
   Current Image: <Slice 12> <image tokens>
   Organ regions: Cerebellum: <bounding box coordinates>, Cerebrum: <bounding box coordinates>, Brainstem: <bounding box coordinates>, . . .
   EHR: <Patient labs and clinical notes>
Actions Left: 100
Next Action:

Continuing with the above example, suppose multimodal generative AI model 222 processes the updated internal state prompt and generates an output healthcare system command that describes an output of “Request physician confirmation of findings: Dr. ______. Suspected subdural hemorrhage, requires emergency intervention”. In this example, generative AI model clinical process 10 provides output healthcare system command associated with alerting the medical professional of the suspected subdural hemorrhage to healthcare system 208 for processing and for obtaining confirmation. As discussed above, the output healthcare system command is converted to a healthcare system-executable command that is executable by a healthcare subsystem that prompts a medical professional for confirmation of the suspected subdural hemorrhage. In this example, a medical professional reviews the medical image content and confirms the suspected subdural hemorrhage. Following the execution of the healthcare system-executable command, generative AI model clinical process 10 updates the internal state prompt as follows:
Task: Indication suspected stroke.
Instruction: Generate findings, coordinate care management.
Quotes:
   Slice 12: “subdural hemorrhage”
Past Actions:
   Change Slice Position to Slice 12
   2. Summarize and reference: “subdural hemorrhage” [slice 12]
   3. Request physician confirmation of findings: Dr. ______. Suspected subdural hemorrhage, requires emergency intervention.
List of input data:
   Current Image: <Slice 12> <image tokens>
   Organ regions: Cerebellum: <bounding box coordinates>, Cerebrum: <bounding box coordinates>, Brainstem: <bounding box coordinates>, . . .
   EHR: <Patient labs and clinical notes>
   Physician confirmation of subdural hemorrhage by Dr. ______.
Actions Left: 100
Next Action:
Continuing with the above example, suppose multimodal generative AI model 222 processes the updated internal state prompt and generates an output healthcare system command that describes an output of “Alert neurologist of confirmed subdural hemorrhage and organize immediate medical attention”. In this example, generative AI model clinical process 10 provides output healthcare system command associated with alerting a neurologist of the now-confirmed subdural hemorrhage to healthcare system 208 for processing and for scheduling immediate medical attention (e.g., scheduling an emergency medical procedure, contacting a primary physician, etc.). As discussed above, the output healthcare system command is converted to a healthcare system-executable command that is executable by a healthcare subsystem that alerts a neurologist of the confirmed subdural hemorrhage. In this example, a neurologist is informed of the subdural hemorrhage and emergency medical attention is requested (e.g., an emergency medical procedure is scheduled and associated individuals receive notifications). Following the execution of the healthcare system-executable command, generative AI model clinical process 10 updates the internal state prompt as follows:
Task: Indication suspected stroke.
Instruction: Generate findings, coordinate care management.
Quotes:
   Slice 12: “subdural hemorrhage”
Past Actions:
   Change Slice Position to Slice 12
   2. Summarize and reference: “subdural hemorrhage” [slice 12]
   3. Request physician confirmation of findings: Dr. ______. Suspected subdural hemorrhage, requires emergency intervention.
   Alert neurologist of confirmed subdural hemorrhage and organize immediate medical attention
List of input data:
   Current Image: <Slice 12> <image tokens>
   Organ regions: Cerebellum: <bounding box coordinates>, Cerebrum: <bounding box coordinates>, Brainstem: <bounding box coordinates>, . . .
   EHR: <Patient labs and clinical notes>
   Physician confirmation of subdural hemorrhage by Dr. ______.
   Scheduled medical procedure
Actions Left: 100
Next Action:

Continuing with the above example, suppose multimodal generative AI model 222 processes the updated internal state prompt and generates an output healthcare system command that describes an output of “Generate Findings <Findings>. End report.”. In this example, generative AI model clinical process 10 provides output healthcare system command associated with recording the findings to a radiology report for the medical image content to healthcare system 208 for processing. As discussed above, the output healthcare system command is converted to a healthcare system-executable command that is executable by a healthcare subsystem that updates the radiologist report concerning the patient. Following the execution of the healthcare system-executable command, generative AI model clinical process 10 completes multi-action task 206.

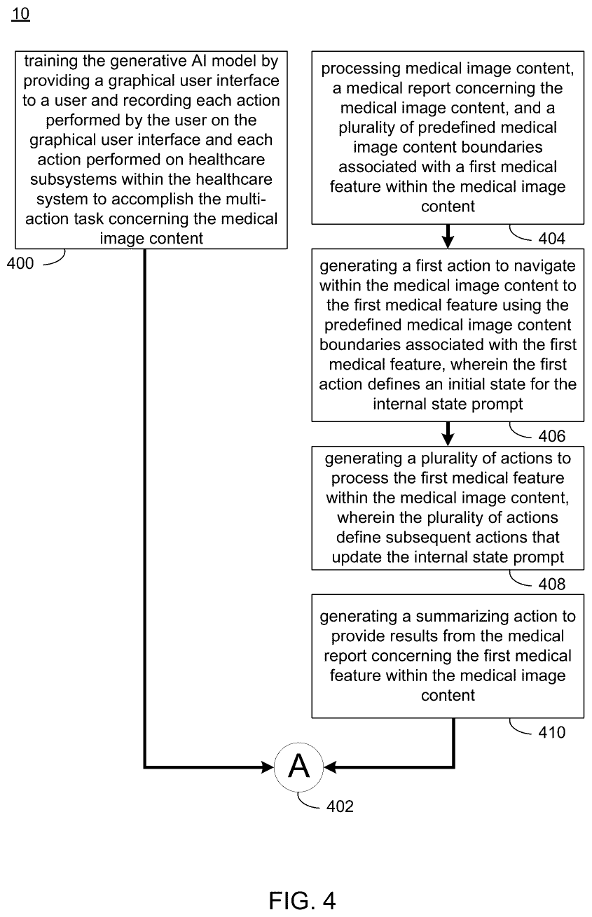

In some implementations and referring also to FIG. 4, generative AI model clinical process 10 trains 400 the generative AI model by providing a graphical user interface to a user and recording each action performed by the user on the graphical user interface and each action performed on healthcare subsystems within the healthcare system to accomplish the multi-action task concerning the medical image content. For example, generative AI model clinical process 10 trains multimodal generative AI model 222 by collecting training data by replacing the multimodal generative AI model 222 with a user leveraging a graphical user interface (GUI) that allows a user to interact with the healthcare system in the same way that a multimodal generative AI model would, and collecting the resulting action sequences from the user (using the GUI). Each command executed by the user is recorded in such a manner that would yield action sequence data that resembles that shown in the above example, except that multimodal generative AI model 222 is replaced with the user. In some implementations, generative AI model clinical process 10 trains 400 multimodal generative AI model 222 by defining user interactions with the GUI and resulting action sequences as actions for multimodal generative AI model 222 to perform to accomplish particular actions. In this example, generative AI model clinical process 10 trains 400 multimodal generative AI model 222 using behavior cloning.

In this example, the conditions or state of the interactions between the user and the medical content define an internal state prompt and each action performed by the user using healthcare subsystems 210 is provided to multimodal generative AI model 222 as a healthcare system-executable command. In this manner, multimodal generative AI model 222 is trained with the user interactions and the state of the medical content at each user interaction to generate healthcare system-executable commands and internal state prompts. As described above, healthcare system-executable commands are mapped to the plurality of predefined healthcare system-executable commands and their associated output healthcare system commands. Accordingly, multimodal generative AI model 222 is trained with output healthcare system commands to generate that result in particular healthcare system-executable commands for respective user inputs that define the internal state prompt for each healthcare system-executable command. As shown in FIG. 4, the training of multimodal generative AI model 222 as described above is used during inference with selections of similar multi-action tasks as covered by the training. This is shown in FIG. 4 as the result of training 400 proceeding to action “A” (e.g., action 402) shown in FIG. 1.

In some implementations and referring again to FIG. 4, generative AI model clinical process 10 trains the generative AI model by: processing 404 medical image content, a medical report concerning the medical image content, and a plurality of predefined medical image content boundaries associated with a first medical feature within the medical image content; generating 406 a first action to navigate within the medical image content to the first medical feature using the predefined medical image content boundaries associated with the first medical feature, wherein the first action defines an initial state for the internal state prompt; generating 408 a plurality of actions to process the first medical feature within the medical image content, wherein the plurality of actions define subsequent actions that update the internal state prompt; and generating 410 a summarizing action to provide results from the medical report concerning the first medical feature within the medical image content. For example, obtaining training data from a user may be cost prohibitive in terms of temporal resources (i.e., the time required to perform training of multimodal generative AI model 222 using a user). Accordingly and as an alternative to collecting training data directly from users, generative AI model clinical process 10 applies a rule-based approach to convert existing “grounded” datasets into action sequences. In this example and in some implementations, generative AI model clinical process 10 trains the generative AI model by applying behavior cloning using observational data associated with a user performing each action of the multi-action task.

For example and in some implementations, generative AI model clinical process 10 processes 404 medical image content as input data: medical image content (e.g., two-dimensional or three-dimensional volume), a medical report concerning the medical image content (e.g., radiological report), and a plurality of predefined medical image content boundaries associated with a first medical feature within the medical image content (e.g., associations between subphrases in the medical report and their physical location in the image as bounding box/cube coordinates). Generative AI model clinical process 10 generates 406 a first action to navigate within the medical image content to the first medical feature using the predefined medical image content boundaries associated with the first medical feature. For example, generative AI model clinical process 10 generates an internal state prompt as shown below:
Task: Indication pulmonary discomfort.
Instruction: Generate findings, coordinate care management.
Quotes: [None]
Past Actions: [None]
List of input data:
   Current Image: <Slice 1> <image tokens>
   Organ regions: Left Lung <bounding box coordinates>, Right Lung <bounding box coordinates>, Heart <bounding box coordinates>, Stomach <bounding box coordinates>, Esophagus <bounding box coordinates>, . . .
   EHR: <Patient labs and clinical notes>
Actions Left: 100
Next Action:

As shown above, the internal state prompt includes a first action beginning with an initial medical image content (i.e., “Slice 1”) that is associated with a medical feature (i.e., an organ). For each medical feature (identified from prior organ segmentation using a healthcare subsystem), generative AI model clinical process 10 generates 408 a plurality of actions by generating an action (and associated healthcare system-executable command) to navigate the currently viewed image to the first image in that organ and iteratively issuing subsequent actions with corresponding healthcare system-executable commands to navigate over all slices in the organ (e.g., “N” actions, where “N” is the total number of images of the medical feature). Upon reaching a medical image content (i.e., slice) that overlaps with any grounded pathology in that medical feature (i.e., organ), generative AI model clinical process 10 generates 410 a summarizing action and associated healthcare system-executable command to provide results from the medical report concerning the first medical feature within the medical image content. For example, generative AI model clinical process 10 generates a healthcare system-executable command to “Summarize and Reference: <ref>” where <ref> is replaced by the text associated with the grounded region for “M” actions where “M” is the number of slices overlapping with the pathology. In other words, generative AI model clinical process 10 generates “M” actions and associated healthcare system-executable commands that direct a healthcare subsystem to summarize and reference each overlap in the pathology. With each healthcare system-executable command, the internal state prompt is updated to reflect what the next internal state prompt will include for a subsequent iteration by multimodal generative AI model 222. In some implementations, if the slices are completed with no pathology, generative AI model clinical process 10 generates 408 a summarizing action and associated healthcare system-executable command indicative of no issues (e.g., by generating a healthcare system-executable command to “Summarize and reference: <organ> normal appearance”). In this example, generative AI model clinical process 10 generates 410 a summarizing action and healthcare system-executable command to provide results from the medical report concerning the first medical feature within the medical image content with indications of slices overlapping with the pathology, or with an indication of normal appearance from the absence overlapping in the pathology.

In some implementations, generative AI model clinical process 10 generates a final action and associated healthcare system-executable command to “Generate findings: <findings>” where <findings> are the “ground truth” findings from the medical report concerning the medical image content (e.g., the radiological report). Using the generated actions (i.e., first action to navigate within the medical image content to the first medical feature using the predefined medical image content boundaries associated with the first medical feature, subsequent actions to navigate within the medical image content to each medical feature using the predefined medical image content boundaries associated with the each respective medical feature, the summarizing actions and healthcare system-executable commands to provide summaries of each action, and the final action and associated healthcare system-executable command to generate the findings from the medical report), generative AI model clinical process 10 trains multimodal generative AI model 222 to process a multi-action task, a first action, medical image content, and a medical report concerning the medical image content to define which actions to perform on the medical image content to perform the multi-action task and produce findings as shown in the medical report concerning the medical image content. In one example, generative AI model clinical process 10 trains multimodal generative AI model 222 using behavior cloning (i.e., supervised learning on observation-action pairs from expert demonstrations). In another example, generative AI model clinical process 10 trains multimodal generative AI model 222 using reward modeling (i.e., where the multimodal generative AI model receives a reward for its responses to given prompts. This reward signal serves as feedback, guiding the multimodal generative AI model to produce desired outcomes). Accordingly, it will be appreciated that generative AI model clinical process 10 trains multimodal generative AI model 222 using various known methods.

As shown in FIG. 4, the training of multimodal generative AI model 222 as described above is used during inference with selections of similar multi-action tasks as covered by the training. This is shown in FIG. 4 as the result of generating 408 a summarizing action proceeding to action “A” (e.g., action 402) shown in FIG. 1. Accordingly, generative AI model clinical process 10 is able to provide multiple manners of training multimodal generative AI model 222 to perform multi-action tasks by providing, in one example, user interactions as recorded in a graphical user interface, and, in another example, by applying the findings of a medical report concerning medical image content to provide the actions needed to identify the findings of the medical report from the medical image content.

### System Overview:

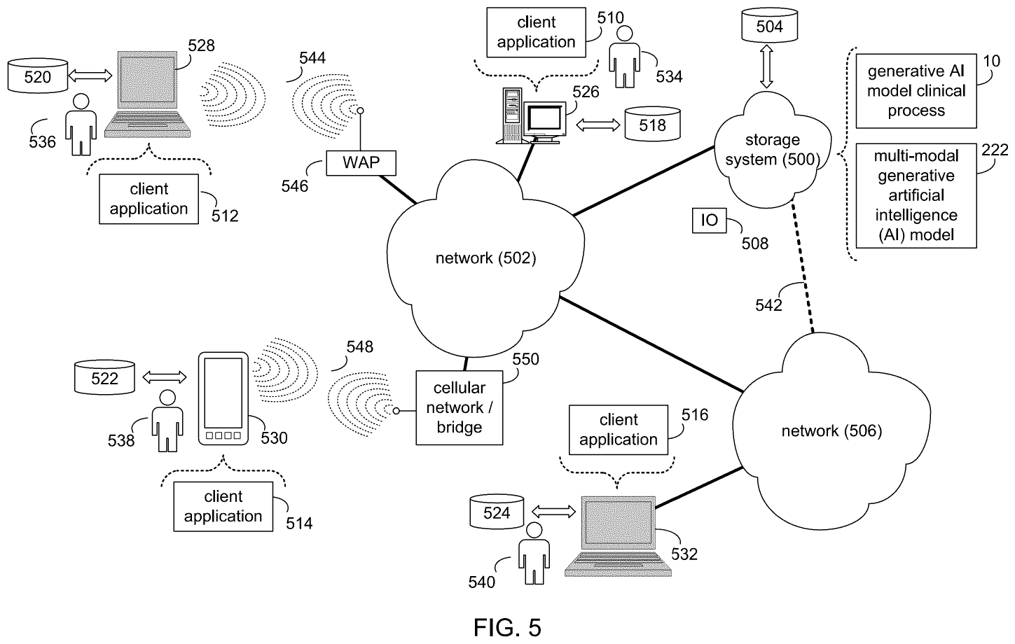

Referring to FIG. 5, a generative AI model clinical process 10 is shown to reside on and is executed by storage system 500, which is connected to network 502 (e.g., the Internet or a local area network). Examples of storage system 500 include: a Network Attached Storage (NAS) system, a Storage Area Network (SAN), a personal computer with a memory system, a server computer with a memory system, and a cloud-based device with a memory system. A SAN includes one or more of a personal computer, a server computer, a series of server computers, a minicomputer, a mainframe computer, a RAID device, and a NAS system.

The various components of storage system 500 execute one or more operating systems, examples of which include: Microsoft® Windows®; Mac® OS X®; Red Hat® Linux®, Windows® Mobile, Chrome OS, Blackberry OS, Fire OS, or a custom operating system (Microsoft and Windows are registered trademarks of Microsoft Corporation in the United States, other countries or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

The instruction sets and subroutines of generative AI model clinical process 10, which are stored on storage device 504 included within storage system 500, are executed by one or more processors (not shown) and one or more memory architectures (not shown) included within storage system 500. Storage device 504 may include: a hard disk drive; an optical drive; a RAID device; a random-access memory (RAM); a read-only memory (ROM); and all forms of flash memory storage devices. Additionally or alternatively, some portions of the instruction sets and subroutines of generative AI model clinical process 10 are stored on storage devices (and/or executed by processors and memory architectures) that are external to storage system 500.

In some implementations, network 502 is connected to one or more secondary networks (e.g., network 506), examples of which include: a local area network; a wide area network; or an intranet.

Various input/output (IO) requests (e.g., IO request 508) are sent from client applications 510, 512, 514, 516 to storage system 500. Examples of IO request 508 include data write requests (e.g., a request that content be written to storage system 500) and data read requests (e.g., a request that content be read from storage system 500).

The instruction sets and subroutines of client applications 510, 512, 514, 516, which may be stored on storage devices 518, 520, 522, 524 (respectively) coupled to client electronic devices 526, 528, 530, 532 (respectively), may be executed by one or more processors (not shown) and one or more memory architectures (not shown) incorporated into client electronic devices 526, 528, 530, 532 (respectively). Storage devices 518, 520, 522, 524 may include: hard disk drives; tape drives; optical drives; RAID devices; random access memories (RAM); read-only memories (ROM), and all forms of flash memory storage devices. Examples of client electronic devices 526, 528, 530, 532 include personal computer 526, laptop computer 528, smartphone 530, laptop computer 532, a server (not shown), a data-enabled, and a dedicated network device (not shown). Client electronic devices 526, 528, 530, 532 each execute an operating system.

Users 534, 536, 538, 540 may access storage system 500 directly through network 502 or through secondary network 506. Further, storage system 500 may be connected to network 502 through secondary network 506, as illustrated with link line 542.

The various client electronic devices may be directly or indirectly coupled to network 502 (or network 506). For example, personal computer 526 is shown directly coupled to network 502 via a hardwired network connection. Further, laptop computer 532 is shown directly coupled to network 506 via a hardwired network connection. Laptop computer 528 is shown wirelessly coupled to network 502 via wireless communication channel 544 established between laptop computer 528 and wireless access point (e.g., WAP) 546, which is shown directly coupled to network 502. WAP 546 may be, for example, an IEEE 802.11a, 802.11b, 802.11g, 802.11n, Wi-Fi®, and/or Bluetooth® device that is capable of establishing a wireless communication channel 544 between laptop computer 528 and WAP 546. Smartphone 530 is shown wirelessly coupled to network 502 via wireless communication channel 548 established between smartphone 530 and cellular network/bridge 550, which is shown directly coupled to network 502.

### General:

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be used. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet.

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, not at all, or in any combination with any other flowcharts depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
generating an internal state prompt with medical content and a multi-action task to perform on a healthcare system;
generating a first output healthcare system command by processing the internal state prompt using a trained multimodal generative artificial intelligence (AI) model;
converting the first output healthcare system command into a first healthcare system-executable command associated with the multi-action task for a first target healthcare subsystem;
generating modified medical content by executing the first healthcare system-executable command on the medical content using the first target healthcare subsystem;
updating the internal state prompt with the modified medical content generated by executing the first healthcare system-executable command and the first output healthcare system command listed as a past action performed during execution of the multi-action task; and
generating additional output healthcare system commands by processing the updated internal state prompt using the trained multimodal generative AI model with the first output health as context for generating the additional output healthcare system commands to perform the multi-action task.

2. The computer-implemented method of claim 1, wherein generating additional output healthcare system commands includes:
generating a second output healthcare system command by processing the updated internal state prompt using the trained multimodal generative AI model;
converting the second output healthcare system command into a second healthcare system-executable command associated with the multi-action task for a second target healthcare subsystem;
generating modified medical content by executing the second healthcare system-executable command on the second target healthcare subsystem; and
updating the internal state prompt with the modified medical content generated by executing the second healthcare system-executable command and the second output healthcare system command listed as a past action performed during execution of the multi-action task.

3. The computer-implemented method of claim 1, wherein the multimodal generative AI model is one of a large multimodal model (LMM) and a large language model (LLM).

4. The computer-implemented method of claim 1, wherein the medical content includes medical image content.

5. The computer-implemented method of claim 1, wherein the multi-action task includes a series of actions to process medical content for a diagnostic-based task.

6. The computer-implemented method of claim 1, further comprising:
training the generative AI model by providing a graphical user interface to a user and recording each action performed by the user on the graphical user interface and each action performed on healthcare subsystems within the healthcare system to accomplish the multi-action task concerning the medical image content.

7. The computer-implemented method of claim 1, further comprising:
training the generative AI model by:
processing medical image content, a medical report concerning the medical image content, and a plurality of predefined medical image content boundaries associated with a first medical feature within the medical image content;
generating a first action to navigate within the medical image content to the first medical feature using the predefined medical image content boundaries associated with the first medical feature, wherein the first action defines an initial state for the internal state prompt;
generating a plurality of actions to process the first medical feature within the medical image content, wherein the plurality of actions define subsequent actions that update the internal state prompt; and
generating a summarizing action to provide results from the medical report concerning the first medical feature within the medical image content.

8. A computing system comprising:
a memory; and
a processor configured to train a multimodal generative artificial intelligence (AI) model for processing an internal state prompt with medical content and a multi-action task to perform on a healthcare system by:
processing medical image content, a medical report concerning the medical image content, and a plurality of predefined medical image content boundaries associated with a first medical feature within the medical image content;
generating a first action to navigate within the medical image content to the first medical feature using the predefined medical image content boundaries associated with the first medical feature, wherein the first action defines an initial state for an internal state prompt;
generating a plurality of actions to process the first medical feature within the medical image content, wherein the plurality of actions define subsequent actions that update the internal state prompt; and
generating a summarizing action to provide results from the medical report concerning the first medical feature within the medical image content.

9. The computing system of claim 8, wherein the processor is further configured to:
generate the internal state prompt with new medical content and a multi-action task to execute on a healthcare system.

10. The computing system of claim 9, wherein the processor is further configured to:
generate a first output healthcare system command by processing the internal state prompt using a trained multimodal generative AI model.

11. The computing system of claim 10, wherein the processor is further configured to:
convert the first output healthcare system command into a first healthcare system-executable command associated with the multi-action task for a first target healthcare subsystem.

12. The computing system of claim 11, wherein the processor is further configured to:
generate modified medical content by executing the first healthcare system-executable command on the medical content using the first target healthcare subsystem.

13. The computing system of claim 12, wherein the processor is further configured to:
update the internal state prompt with the modified medical content generated by executing the first healthcare system-executable command and the first output healthcare system command listed as a past action performed during execution of the multi-action task.

14. The computing system of claim 13, wherein the multimodal generative AI model is one of a large multimodal model (LMM) and a large language model (LLM).

15. A computer program product residing on a computer readable medium having a plurality of instructions stored thereon which, when executed by a processor, cause the processor to perform operations comprising:
generating an internal state prompt with medical image content and a diagnostic multi-action task to perform on a healthcare system;
generating modified medical image content by processing the internal state prompt using a trained multimodal generative artificial intelligence (AI) model;
converting the first output healthcare system command into a first healthcare system-executable command associated with the multi-action task for a first target healthcare subsystem;
generating modified medical image content by executing the first healthcare system-executable command on the medical content using the first target healthcare subsystem; and
updating the internal state prompt with the modified medical image content generated by executing the first healthcare system-executable command and the first output healthcare system command listed as a past action performed during execution of the multi-action task.

16. The computer program product of claim 15, wherein the operations further comprise:
generating a second output healthcare system command by processing the updated internal state prompt using the trained multimodal generative AI model;
converting the second output healthcare system command into a second healthcare system-executable command associated with the multi-action task for a second target healthcare subsystem;
generating modified medical content by executing the second healthcare system-executable command on the second target healthcare subsystem; and
updating the internal state prompt with the modified medical content generated by executing the second healthcare system-executable command and the second output healthcare system command listed as a past action performed during execution of the multi-action task.

17. The computer program product of claim 15, wherein the multimodal generative AI model is one of a large multimodal model (LMM) and a large language model (LLM).

18. The computer program product of claim 15, wherein the operations further comprise:
training the generative AI model by providing a graphical user interface to a user and recording each action performed by the user on the graphical user interface and each action performed on healthcare subsystems within the healthcare system to accomplish the multi-action task concerning the medical image content.

19. The computer program product of claim 15, training the generative AI model by:
processing medical image content, a medical report concerning the medical image content, and a plurality of predefined medical image content boundaries associated with a first medical feature within the medical image content;
generating a first action to navigate within the medical image content to the first medical feature using the predefined medical image content boundaries associated with the first medical feature, wherein the first action defines an initial state for the internal state prompt;
generating a plurality of actions to process the first medical feature within the medical image content, wherein the plurality of actions define subsequent actions that update the internal state prompt; and
generating a summarizing action to provide results from the medical report concerning the first medical feature within the medical image content.

20. The computer program product of claim 15, wherein converting the first output healthcare system command into the first healthcare system-executable command includes identifying a predefined healthcare system-executable command for the first output healthcare system command from a plurality of predefined healthcare system-executable commands.
